(19) United States
(12) Patent Application Publication
(10) Pub. No.: US 2015/0041615 A1
(43) Pub. Date: Feb. 12, 2015

(54) EXTRA CORPOREAL LIFE SUPPORT LEVELLING PLATFORM

(71) Applicant: THE HOSPITAL FOR SICK CHILDREN, Toronto (CA)

(72) Inventors: Jamil Lati, Aurora (CA); Carl A. Granger, Scarborough (CA); Eric Peter Niles, Hamilton (CA)

(73) Assignee: THE HOSPITAL FOR SICK CHILDREN, Toronto (CA)

(21) Appl. No.: 14/384,124

(22) PCT Filed: Mar. 11, 2013
(86) PCT No.: PCT/CA2013/050175

§ 371 Date: Sep. 9, 2014

Related U.S. Application Data
(60) Provisional application No. 61/608,997, filed on Mar. 9, 2012.

(51) Int. Cl. A61B 19/00; A61M 1/16

(52) U.S. Cl. CPC A61B 19/26; A61M 1/1698; A61M 2209/086; A61B 2019/268
USPC 248/543

(57) ABSTRACT

The present invention provides a leveling platform for an i LA®/external lung device which can be patient adjusted. The platform includes upper and lower planar support members pivotally attached to each other along one side of each member and the opposed side includes an adjustment mechanism for pivoting the top support member with respect to the bottom support members to provide a level adjustment. The top support member includes a retaining mechanism for holding an i LA® ECLS device and level indicators to indicate whether or not the top support member is level. The bottom support member rests on a surface such as a bed or chair to which the platform can be secured. Once the ECLS device is connected to the patient and supported in the ECLS device is mounted on the top surface of the leveling platform and if required, the adjustment mechanism is engaged by the clinician or patient to level the ECLS device. The leveling platform safely secures an i LA®/ECLS device to a bed, chair, wheelchair, table, pole, walker or cart. The leveling platform disclosed herein addresses the current clinical requirements and safety issues associated with the i LA®/ECLS devices.

### FIELD

The present invention relates generally to a holder for an external lung device which includes a leveling mechanism so it can be patient and/or care giver adjusted.

### BACKGROUND

The NovaLung® iLA® membrane ventilator and other extracorporal life support (ECLS) devices provide many advantages in the treatment of patients with critical dysfunction of the cardiopulmonary system. The Novalung® Interventional Lung Assist (iLA®) device is a membrane ventilator that allows for oxygen and carbon dioxide gas exchange to occur by simple diffusion. It is an arterio-venous pumpless ventilation support device and therefore it is driven by patient's cardiac output. A cannula is placed to divert deoxygenated blood to the iLA®. The blood flows through the membrane, and returns via a second cannula to the patient. Gas transfer takes place through the membrane by diffusion of O₂ across the membrane to displace CO₂ and exhaust from the bottom of the device. The primary function of the iLA® is CO₂ removal however it also supplements O₂ support.

Unfortunately, the main disadvantage of these external systems is the common requirement to leave patients confined to their hospital bed. One, for the reason of safety, and two for the reason of resource availability. The adverse events that can occur include accidental decannulation which occur if the iLA® is tipped over, falls, or is ineffectively repositioned. Decannulation can also occur if the iLA®/ECLS device is inadvertently pulled away from the patient (example if patient is sitting in a chair, and the iLA®/ECLS is place on a table or the bed, the table/bed/chair can be accidentally moved).

Another problem that can arise is air being observed in the blood flow tubing connecting the patient to the device which may occur because of lack of exhaust or having the iLA® unleveled or tilted. Another problem is acute failure which may occur due to either of the above-listed reasons, including poor positioning of the iLA®, or the collection of condensation below the device which prevents exhaust.

Recent trends in Intensive care practices focus on early mobilization of patients on advance life support technologies in order to prevent the detrimental consequences of prolonged bed rest. These include muscle wasting, bed sores, inflexibility, decreased exercise tolerance and endurance, psychosocial deterioration due to confinement, and most significantly an increased risk of pulmonary sequelae such as pneumonia and lung segment collapse (atelectasis).

Several ICU mobility strategies have been attempted; however, these current strategies do not allow for safe management of the very acute ICU patient connected to a iLA®/ECLS system.

Thus, there is a need for a leveling platform for an external lung device which can be patient adjusted.

### SUMMARY

The present invention provides a leveling platform for an external lung device which can be patient adjusted. The platform includes upper and lower planar support members pivotally attached to each other along one side of each member and the opposed side includes an adjustment mechanism for pivoting the top support member with respect to the bottom support members to provide a level adjustment. The top support member includes a retaining mechanism for holding an ECLS device and level indicators to indicate whether or not the top support member is level. The bottom support member rests on a surface such as a bed or chair or adjustable mounting arm to which the platform can be secured. Once the ECLS device is connected to the patient and supported in the ECLS device is mounted on the top surface of the leveling platform and if required, the adjustment mechanism is engaged by the clinician or patient to level the ECLS device.

Thus an embodiment of the leveling platform comprises:

an upper substantially planar support member having an upper surface and a retaining mechanism for releasibly holding a holder for an extra corporeal life support membrane on said upper surface;

a lower substantially planar support members, said upper and lower support members being pivotally attached together so that said upper planar support member can pivot with respect to the lower planar support member;

at least one level indicator mounted on said upper planar support member for showing deviations from horizontal of said upper support member; and

adjustment mechanism for pivotally adjusting said upper support member with respect to said lower support member for adjusting an angle between said upper and lower support members for leveling the upper member to the horizontal.

A further understanding of the functional and advantageous aspects of the invention can be realized by reference to the following detailed description and drawings.

### BRIEF DESCRIPTION OF THE DRAWINGS

Preferred embodiments of the invention will now be described, by way of example only, with reference to the drawings, in which:

FIG. 1 shows perspective view of a leveling platform assembled with a holder for an external lung device (shown as the NovaLung® iLA® membrane ventilator “holder”) constructed in accordance with the present invention;

FIG. 2 is a perspective view of the combination of FIG. 1 but taken from a different position that FIG. 1;

FIG. 3 is a disassembled view of the combination of FIGS. 1 and 2 in a first configuration;

FIG. 4 is a disassembled view of the combination of FIGS. 1 and 2 in a second configuration;

FIG. 5 is an perspective view of the device similar to FIG. 1;

FIG. 6 is a top view of the device of FIG. 5 taken along arrow 6;

FIG. 7 is a view of the device of FIG. 5 taken along arrow 7;

FIG. 8 is a view of the device of FIG. 5 taken along arrow 8; and

FIG. 9 is a view of the device of FIG. 5 taken along arrow 9.

### DETAILED DESCRIPTION

Without limitation, the embodiments described herein are directed to a holder for an external lung device which includes a leveling mechanism so it can be patient adjusted. As required, embodiments of the present invention are disclosed herein. However, the disclosed embodiments are merely exemplary, and it should be understood that the invention may be embodied in many various and alternative forms.

The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to a holder for an external lung device which includes a leveling mechanism.

As used herein, the Novalung ® Interventional Lung Assist (iLA®) device refers to a membrane ventilator that allows for oxygen and carbon dioxide gas exchange to occur by simple diffusion.

As used herein, ECLS means an extracorporeal life support which is a device that provides one or more aspects of the following support: Membrane ventilation that exchanges oxygen for carbon dioxide gas; Circulatory/cardiac output assistance through an external drive device.

Broadly, the present invention provides a holder for an external lung device which can be patient adjusted. In view of the fact that future directions intensive care unit (ICU) standards of care include the goal of maximizing the mobility of patients, the present disclose provides a leveling platform which safely secures an iLA®/ECLS device to a bed, chair, wheelchair, table, pole, walker or cart. The leveling platform disclosed herein addresses the current clinical requirements and safety issues associated with the iLA®/ECLS devices.

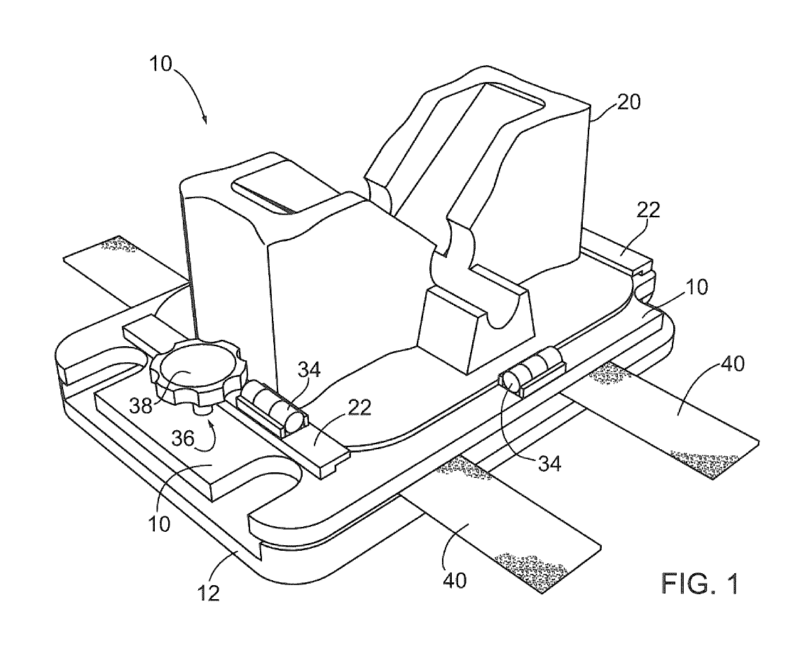

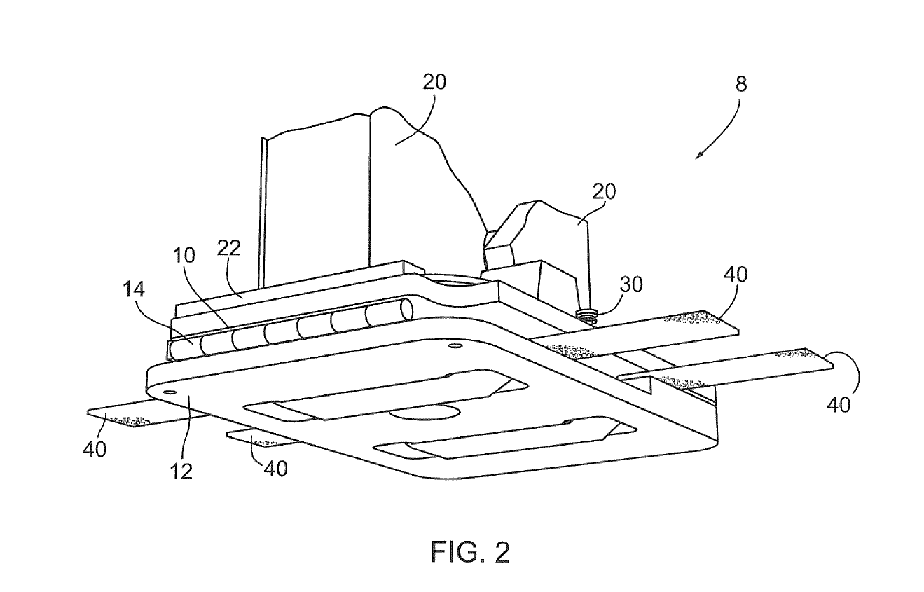

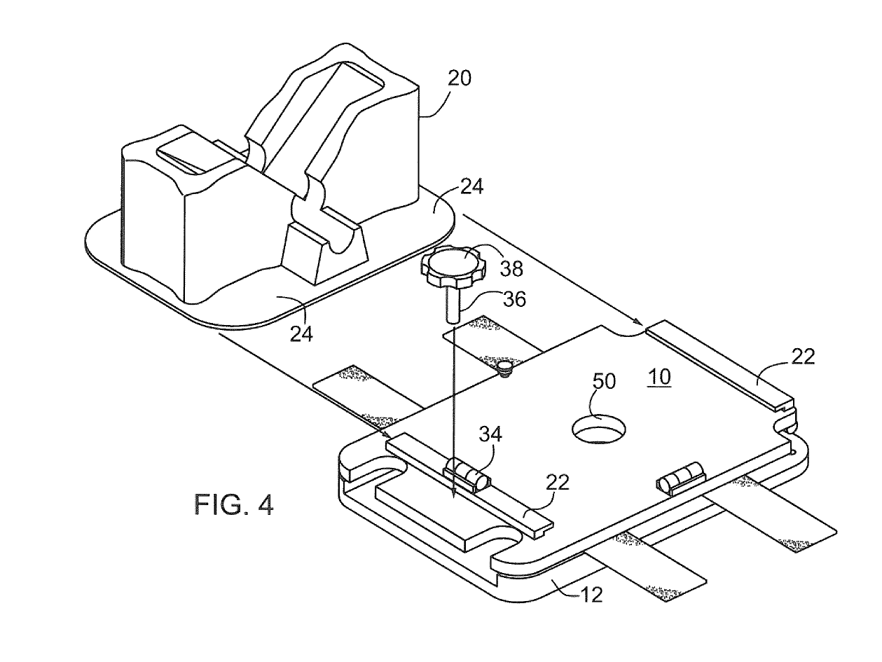

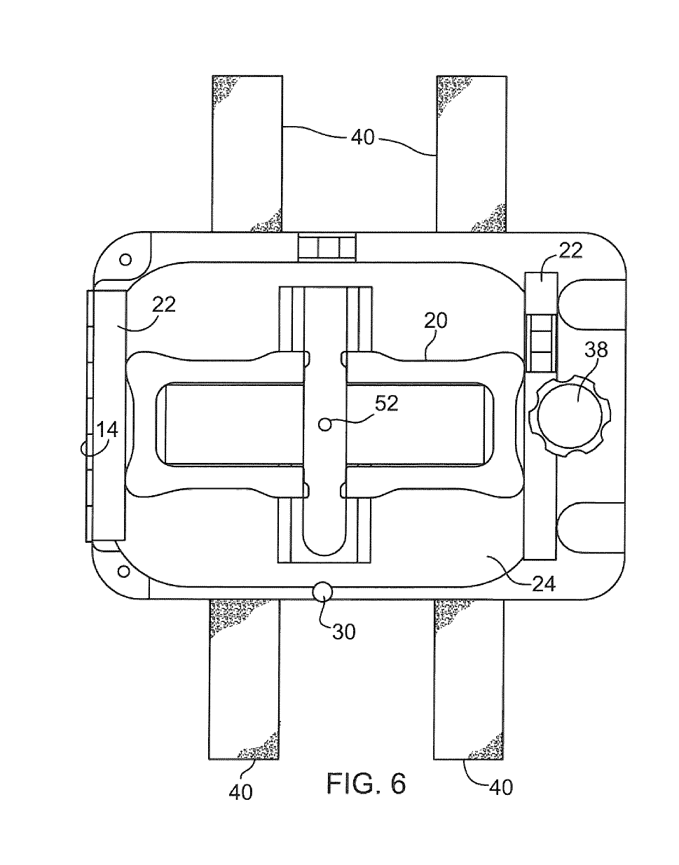

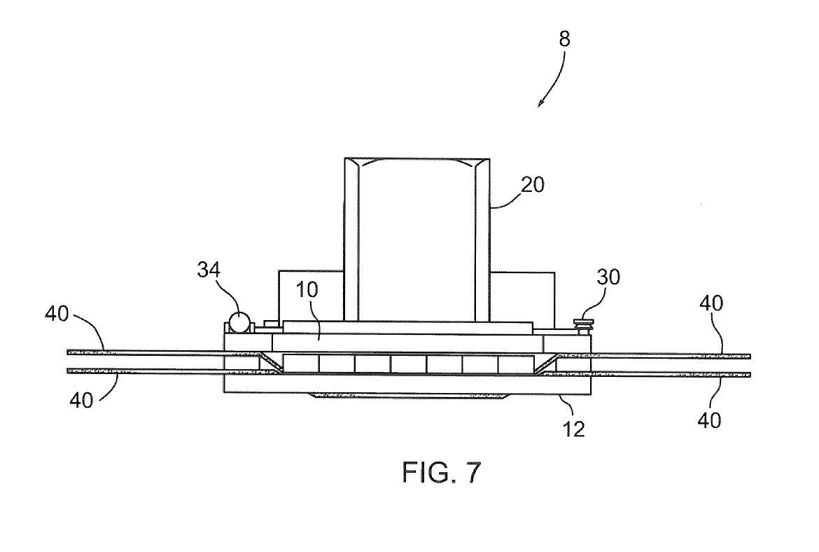

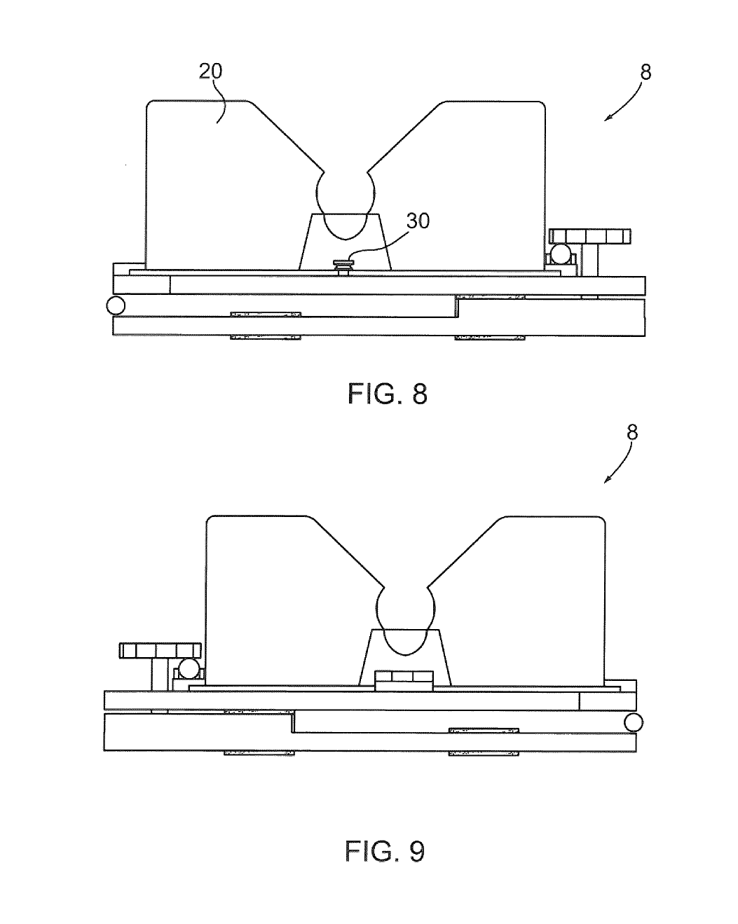

More particularly, the present invention discloses a leveling platform/holder for an extra corporeal life support membrane which can be leveled by a clinician or the patient themselves. Referring to FIGS. 1 to 9, a holder 8 constructed in accordance with the present invention includes an upper planar support member 10, a lower planar support member 12, with support members 10 and 12 being pivotally attached along one end of each member by way of a longitudinal hinge 14. Planar support member 12 is preferably flat so it can rest on flat surfaces such as a bed mattress.

It will be understood that the leveling platform is not restricted to the embodiment shown in FIGS. 1 to 9. Specifically, instead of a hinge pivotally connecting the upper and lower planar members, it will be appreciated that embodiments may be constructed using an articulated joint at the longitudinal end, or the center of the upper planar support member, or a fulcrum place at either horizontal end of the upper planar support member. Conversely, a curved track may be placed at either horizontal end of the lower planar support member and is connected to the upper planar support member in such a way that the upper planar support member is able to adjust its angle by being positioned along the track. In all methods of angle/level adjustment the mechanism can be adjusted through mechanical, pneumatic, self-locking, or electronic methods.

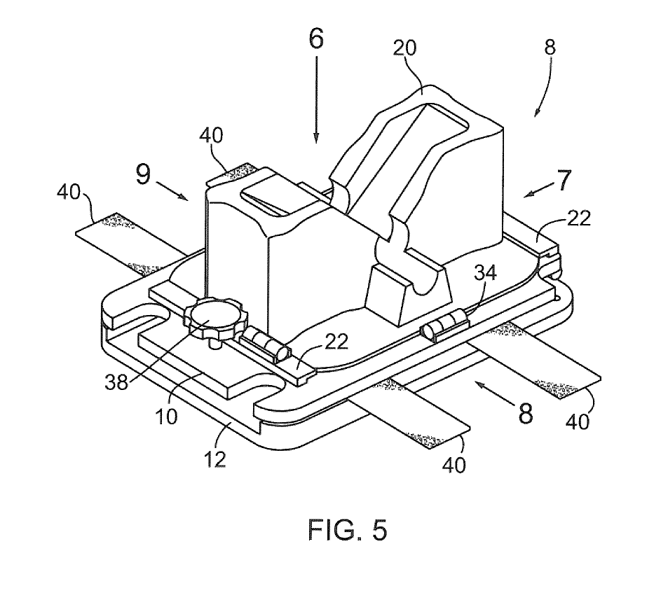

Upper planar support member 10 is planar and is configured with a retaining mechanism to releasibly hold an extra corporeal life support (ECLS) holder 20 (depicted as the NovaLung® iLA® membrane ventilator “holder”) on its upper surface. The retaining mechanism includes upper top surface of support member 10 having a bracket 22 which extends around the peripheral edge of three (s) sides of support member 10. When being secured to the holder 10, the (ECLS) holder 20 is slid into engagement with upper member 10 by being slid into the fourth open side (where bracket 22 is absent) so that that the flat peripheral edges 24 of ECLS holder 20 are firmly between bracket 22 and the upper surface of upper planar member 10 (best seen in FIGS. 1 and 5. Once in place a thumb screw 30 is threaded into a threaded hole in upper planar support member 10 on the open fourth side of the member 10 thereby retaining ECLS holder 20 in place on the top surface of member 10.

It will be appreciated that there are configurations that could be used for retaining the (ECLS) holder 20. For example, in another embodiment the ECLS holder may also be secured through a molded surface of the upper planar member which conforms to the ECLS holder for quick attachment and release. The ECLS holder may also be secured to the upper planar member through a quick clip or “VELCRO” system made of a rigid or semi-rigid material which allows effective cleaning. Alternatively, the upper planar member may be formed as one piece to incorporate the ECLS holder into it.

Holder 10 includes at least one level indicator 34 mounted on upper planar support member 10, and may include two (2) as shown in FIG. 1 with the level indicators 34 being mounted perpendicular to each other. The level indicators 34 are used for showing deviations from horizontal of upper support member 10.

Level indicators may be 34 may be bubble level(s) such as used in carpenter's levels or they may be electronic leveling devices with or without angle description and may incorporate an alarm (visual, audio or both) when the upper planar member 10 is not level.

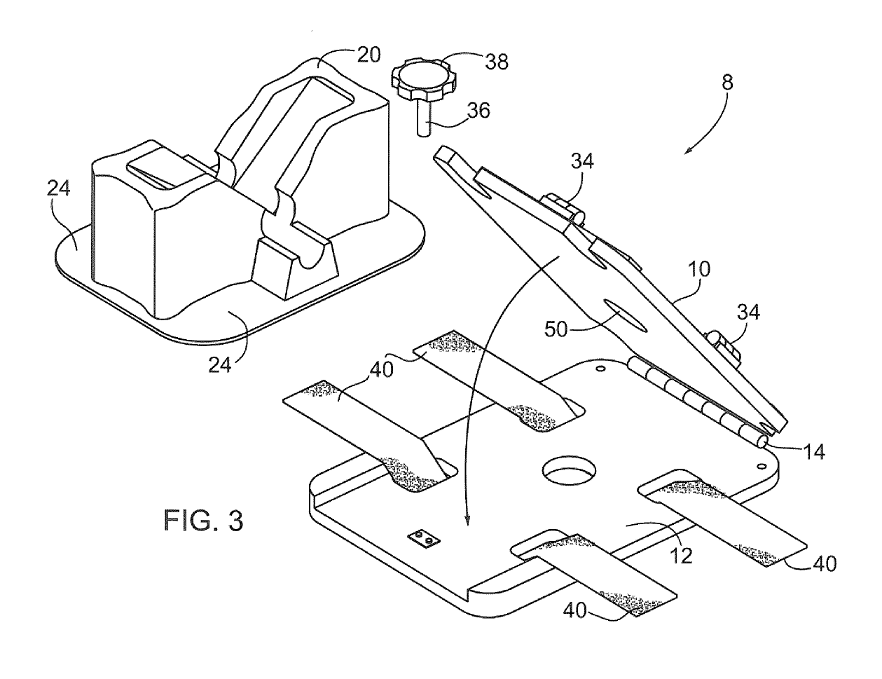

Holder 10 includes an adjustment mechanism for pivotally adjusting the upper support member 10 with respect to the lower support member 12 for adjusting an angle between the upper and lower support members for leveling the upper member 10 to the horizontal. An exemplary mechanism is shown in FIG. 3 and includes a threaded bolt 36 threaded through threads in the end portion of upper support member 10 at the opposite end to the hinge 14. A handle 38 is attached at one end of the bolt 36 which is turned clockwise or counterclockwise. The other end of the bolt 36 bears against the upper surface of lower support member 12 so that as the bolt 36 is hand turned the upper support member 10 pivots about the hinge connection with respect to lower support member 12. The bolt 36 is turned until the level indicators 34 show the upper support member 10 is horizontal.

It will be appreciated that there are other configurations that could be used for the adjustment mechanism instead of the threaded bolt. For example, the adjustment of the upper planar member angle may alternately achieved through a quick slide tab, a notched lever, a pneumatic track, or through the inherent function of the pivotal attachment to the upper planar member.

Holder 10 includes a pair of straps 40 which have quick release couplings for securing the holder stationary on a bed or other flat surface.

The straps 40 can slide with respect to lower support member 10 so the length of the straps on each side of the holder 10 can be varied, depending on how far the attachment points are (such as bed frame, restraint brackets, or rails). In this way the position of holder 10 can readily and easily changed on the surface on which it is located, usually dictated by patient needs.

Since the sites of cannulation may differ between patients (femoral, mediastinal, and potentially upper-thoracic/neck with new cannula available), the leveling platform disclosed herein is configured to be secured by several accessories which may accommodate a wide variety of positions.

The leveling platform provides a secure, flat and level surface for the iLA®/ECLS. An embodiment of the leveling platform secures the membrane with slide tables and a locking pin. To ensure that the iLA®/ECLS remains level at all times, at least one leveling monitor is affixed to multiple planes. To accommodate and promote patient repositioning, activity and mobilization, the leveling platform incorporates a tilting feature to adjust the angle and level the iLA®/ECLS even when the surface is at a tilt. This is done in an embodiment through a tilt adjuster and hinge connecting the top and bottom support members of the leveling platform.

To ensure the leveling platform does not accidentally fold open, the tilt adjuster locks the current “set” position and has a maximum available tilt.

To accommodate the need for air circulation and exhaust at the base of the iLA®/ECLS, the leveling platform 10 includes an exhaust vent/drain 50 incorporated in its top surface as best seen in FIG. 3 which aligns with a hole 52 in holder 20 an a comparable hole 54 in lower support member 12. This allows for the exhaust of CO₂ and allows the condensation which collects at the bottom of the iLA®/ECLS to drain, thus promoting proper functioning. Alternatively, this can be achieved through a continuous positive or negative pressure flow or air with a temperature regulator.

To ensure the leveling platform remains securely affixed to the bed, slots have been incorporated into the lower planar support member of the leveling platform. These slot will accommodate a strapping system and a track system that ensures the iLA®/ECLS can be repositioned safely and effectively. This will prevent the iLA®/ECLS from tipping or falling off the bed, and will promote the prevention of bed sores, and other sequelae of not repositioning patients frequently. Alternatively, the lower planar member can be secured to a track or strapping system via clips, buckles, pins or magnets.

To ensure the ability to safely mobilize patients out of bed, the bottom surface of the planar lower member of the leveling platform includes bolts to affix the leveling platform to an adjustable arm/clamp which can attach the leveling platform to a chair, wheelchair, table, pole or many other surfaces. This provides the iLA®/ECLS user to maximize their quality of life, mobility, ease of care, and rehabilitation. In addition, the leveling platform also has attachment point(s) for an accompanying atmospheric pressure and relative humidity sensor.

As used herein, the terms “comprises”, “comprising”, “includes” and “including” are to be construed as being inclusive and open ended, and not exclusive. Specifically, when used in this specification including claims, the terms “comprises”, “comprising”, “includes” and “including” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

1. A leveling platform for an LAO/external lung device, comprising:
an upper substantially planar support member having an upper surface and a retaining mechanism for releasibly holding a holder for an extra corporeal life support membrane on said upper surface;
a lower substantially planar support members, said upper and lower support members being pivotally attached together so that said upper planar support member can pivot with respect to the lower planar support member;
at least one level indicator mounted on said upper planar support member for showing deviations from horizontal of said upper support member; and
adjustment mechanism for pivotally adjusting said upper support member with respect to said lower support member for adjusting an angle between said upper and lower support members for leveling the upper member to the horizontal.

2. The leveling platform according to claim 1 wherein said planar upper support member includes a hole therein positioned such that it is aligned with a hole present in the holder for the extra corporeal life support membrane to allow ventilation and air circulation in a bottom of the holder.

3. The leveling platform according to claim 1 wherein said upper and lower planar support members are pivotally attached together along an edge of said upper and lower planar support members by a hinge.

4. The leveling platform according to claim 1 including at least one elongate strap having a sufficient length so that one end of the strap can be secured to a first bed rail and the other end of the strap can be secured to a second bed rail.

5. The leveling platform according to claim 4 wherein said at least one elongate strap is a pair of elongate straps.

6. The leveling platform according to claim 4 wherein said at least one elongate strap is slidably adjustable with respect to the lower planar support member.

7. The leveling platform according to claim 1 wherein said at least one level indicator is two level indicators mounted perpendicular to each other on the upper planar support member.

8. The leveling platform according to claim 4 wherein the upper planar support member has first and second ends, said upper planar support member being pivotally attached to the lower planar support member at said first end, said adjustment mechanism including a threaded nut threaded through the second of said upper planar support member with an end of the nut bearing against an upper surface of the lower planar support member and configured such that hand rotation of the threaded nut pivots the upper planar support member with respect to the lower planar support member.

9. The leveling platform according to claim 1, including a sensor mounted on said upper planar support member and positioned for sensing a relative humidity and pressure in a vicinity of an extra corporeal life support membrane mounted in said leveling platform.

10. The leveling platform according to claim 1, wherein the holder for holding an extra corporeal life support membrane is a molded plastic NovaLung® iLA® membrane ventilator holder.

11. The leveling platform according to claim 1 The leveling platform according to any one of claims 1 to 10 wherein said at least one level indicator is an electronic level indicator.

12. The leveling platform according to claim 1 wherein said at least one level indicator is a bubble level indicator.

13. The leveling platform according to claim 2 wherein said upper and lower planar support members are pivotally attached together along an edge of said upper and lower planar support members by a hinge.

14. The leveling platform according to claim 2 including at least one elongate strap having a sufficient length so that one end of the strap can be secured to a first bed rail and the other end of the strap can be secured to a second bed rail.

15. The leveling platform according to claim 14 wherein said at least one elongate strap is a pair of elongate straps.

16. The leveling platform according to claim 14 wherein said at least one elongate strap is slidably adjustable with respect to the lower planar support member.

17. The leveling platform according to claim 2 wherein said at least one level indicator is two level indicators mounted perpendicular to each other on the upper planar support member.

18. The leveling platform according to claim 3 including at least one elongate strap having a sufficient length so that one end of the strap can be secured to a first bed rail and the other end of the strap can be secured to a second bed rail.

19. The leveling platform according to claim 3 wherein said at least one level indicator is two level indicators mounted perpendicular to each other on the upper planar support member.

20. The leveling platform according to claim 5 wherein the upper planar support member has first and second ends, said upper planar support member being pivotally attached to the lower planar support member at said first end, said adjustment mechanism including a threaded nut threaded through the second of said upper planar support member with an end of the nut bearing against an upper surface of the lower planar support member and configured such that hand rotation of the threaded nut pivots the upper planar support member with respect to the lower planar support member.
